(19) United States
(12) Plant Patent Application Publication
(10) Pub. No.: US 2009/0260118 P1
(43) Pub. Date: Oct. 15, 2009

(54) Redbud tree (Cercis canadensis) variety named 'Gold Crown'

(50) ;

(76) Inventor: Leonard O'Dell Miller, Grove, OK (US)

Correspondence Address: GUY V. MANNING, 1407 TEXAS STREET, SUITE 102, FORT WORTH, TX, 76102, US

(21) Appl. No.: 12/082,399

(22) Filed: Apr. 10, 2008

(51) Int. Cl. A01H 5/00

(52) U.S. Cl. PLT/216

(57) ABSTRACT

A novel variety of the redbud tree Cercis canadensis was discovered in Northern Oklahoma. It exhibits vibrant whitish-yellow spring colors, often mottled (variegate) with unstable green and white chimera which may be reversed. The whitish-yellow leaves cover eighty (80%) percent of the spring tree growth, giving the tree the appearance of having a golden crown. The variety known as ‘Gold Crown’ also branches readily, resulting in relatively full, spreading foliage even in young trees.

### LATIN NAME OF THE GENUS AND SPECIES AND THE VARIETY DENOMINATION OF THE PLANT CLAIMED

Cercis canadensis ‘Gold Crown’

### BACKGROUND OF THE INVENTION

1. Field of the Invention

This invention relates generally to a new and distinct variety of decorative redbud tree known generally by the scientific name Cercis canadensis. I discovered it growing in a cultivated area in my yard in Grove, Okla., in 2001. I have given it the name ‘Gold Crown’ because of its noticeable and distinctive spring and summer foliage.

No federally sponsored research or development was involved in this invention.

2. Background

Redbud trees provide striking early blooms highly noticeable in deciduous forests while their neighbors still are masked in the gray of winter. Thus, redbud trees represent one of the first harbingers of spring. Once their blooms disappear, however, redbud trees become almost unnoticeable amid their neighbors because they assume a dull green color. A redbud tree which retains a showy nature throughout its foliage period would be attractive by contrast.

The Cercis canadensis redbud tree, commonly called “Eastern” redbud, is a widely grown ornamental and naturally occurring tree with a wide habitat in the southern and eastern United States. It does exceptionally well in the warm climates. It is generally characterized by spreading branches with heart-shaped, simple leaves replacing pink and magenta early spring flower clusters along its branches.

The instant novel variety of redbud tree now discovered has size and shape characteristics similar to other Eastern Redbud trees, but displays a showy, whitish-yellow foliage in early spring which causes it to appear to be wearing a golden crown. Spring foliage occasionally includes mottled (variegate) leaves with unstable chimera which may be reversed in come cases. The showy foliage persists into summer and fall.

### SUMMARY OF THE INVENTION

The novel cultivar of the present invention, named Cercis canadensis ‘Gold Crown,’ is believed to be a variant of Cercis canadensis but distinguished by striking coloration variations. Specifically:

(a) new growth exhibits distinctive, white or light yellow, often variegated coloration of its leaves;

(b) during summer, the white leaves turn yellow, creating a crown-like appearance on the new growth portions of the tree.

(c) more vigorous branching in young trees, resulting in full, spreading foliage at a young tree age.

### BRIEF DESCRIPTION OF THE DRAWINGS

FIG. 1 is a photographic view of a mature original tree in spring to early summer, exhibiting the golden crown effect on its new growth.

FIG. 2 is a photographic view of the trunk of the mature original tree of FIG. 1.

FIG. 3 is a photographic view of the tree of FIG. 1 in summer, exhibiting full, mature foliage (against a white backdrop to set it apart from the woodsy background).

FIG. 4 is a photographic view of a group of branches with the new spring growth of the tree of FIG. 1.

FIG. 5 is a photographic close-up view of one branch of the tree of FIG. 1 and the distinctive leaf variegated coloration.

FIG. 6 is a photographic close-up view of the summer leaf and ‘golden crown’ effect on the tree of FIG. 1.

FIG. 7 is a photographic close up of a new, late spring growth color variation (golden, variegated, with chimera) of the tree of FIG. 1.

FIG. 8 is a photographic close-up view of the major branches of the mature original tree of FIG. 1.

FIG. 9 is a photographic view in winter time of the mature original tree of FIG. 1 without foliage and showing overall trunk and branching structure.

FIG. 10 is a close-up view of the winter time view as in FIG. 8 view of the mature original tree of FIG. 1.

### DETAILED DESCRIPTION OF THE INVENTION

The instant variety of redbud tree was discovered as a seedling growing among other redbud trees of the Cercis canadensis species in my front yard near a nursery in Grove, Okla., in 2001. It was successfully asexually reproduced in my yard by bud grafting onto Cercis canadensis seedlings and by semi-hardwood cuttings at the nursery. The novel characteristics are reliably transmitted to succeeding generations.

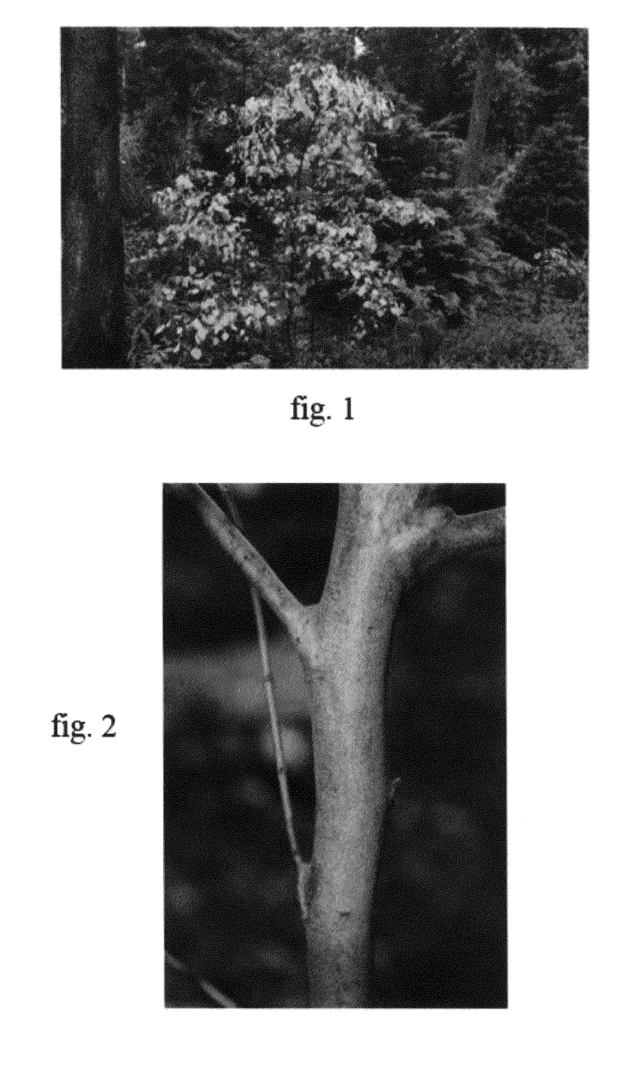

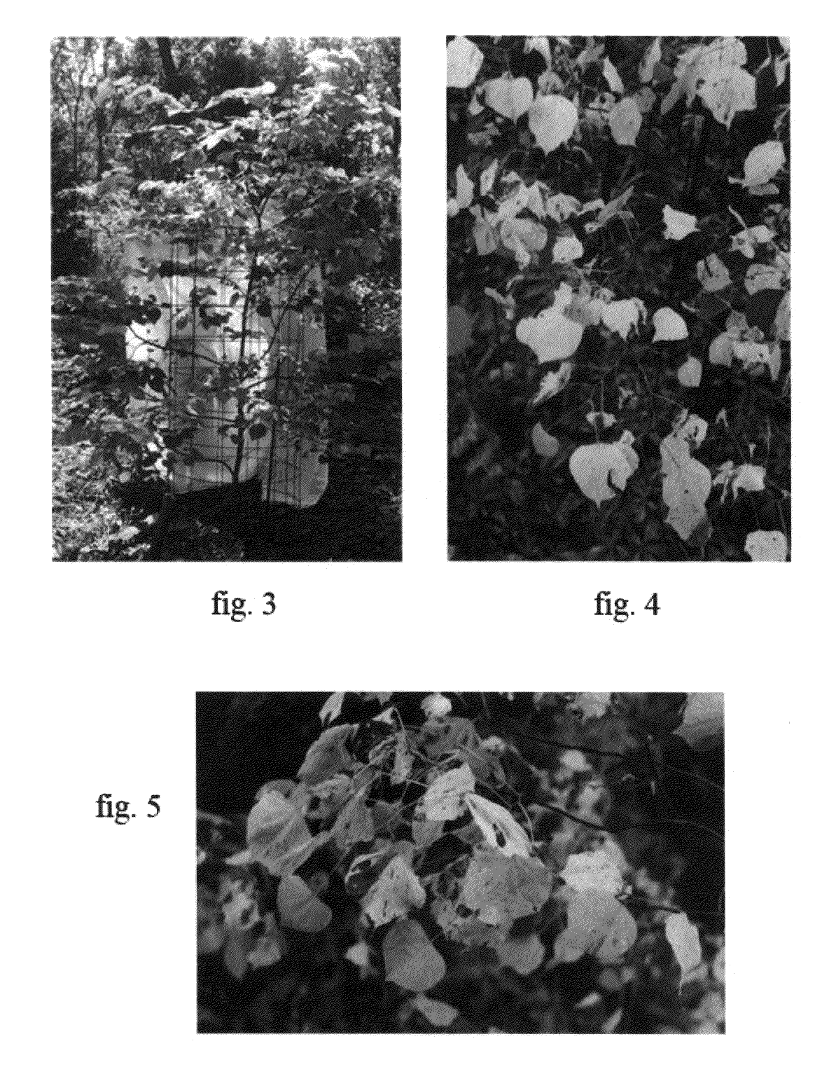

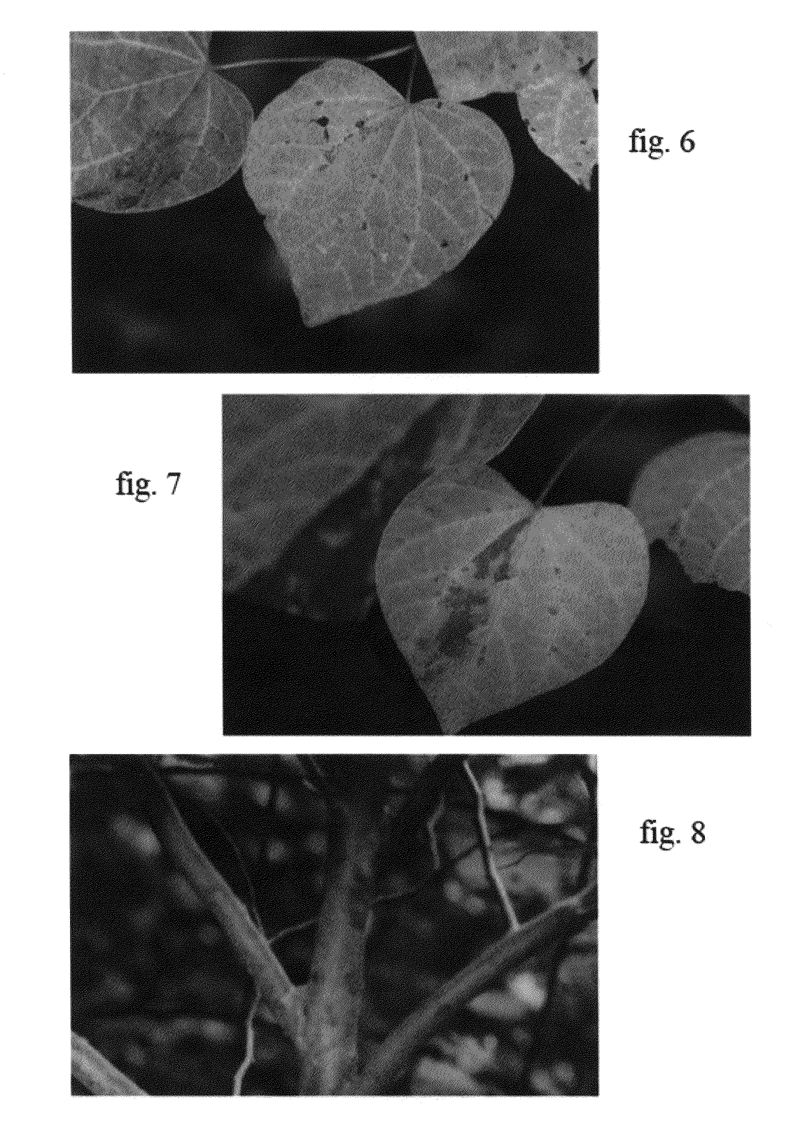

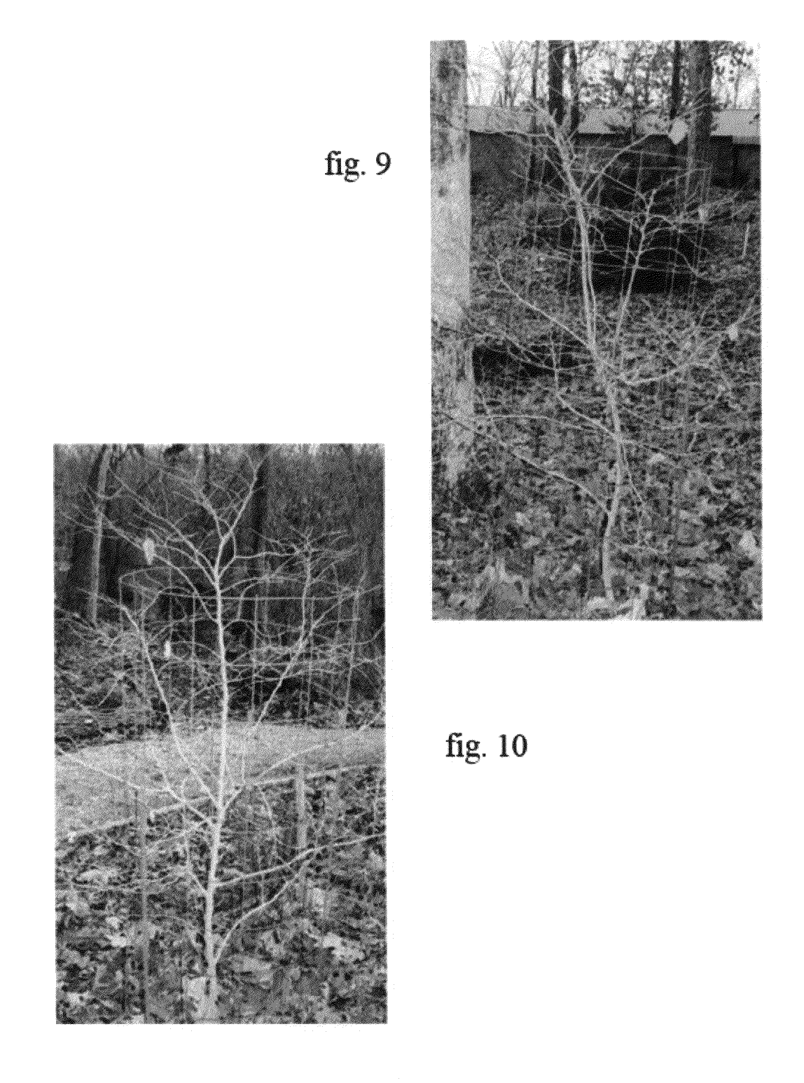

The following specific description of the instant redbud variety details its unique characteristics. Color terminology refers to The Royal Horticultural Society (R.H.S.) Colour Chart.
Origin: A tree of unknown origin, Gold Crown is similar to other Cercis canadensis varieties except for spring and summer leaf coloration. In particular, the spring leaf coloration exhibits whitish-yellow (RHS 145A) overtones, occasionally having mottled (variegate) green and white chimera which are unstable and may be reversed. Summer leaf coloration changes to light yellow (RHS 145C) for substantially 80% of leaves, while the other 20% turn the dull green typical of Cercis canadensis, resulting in a canopy of yellow providing the appearance of a tree wearing a crown. Fall coloration is substantially the same yellow (RHS 145C) as typical Cercis canadensis. Leaf shape matches the Cercis canadensis profile (simple, cordate, acuminate), differing most noticeably by their size, which are two (2″) inches wide and long, whereas the typical Cercis canadensis is three (3″) inches to four (4″) inches wide and long. Branching is active in young trees, presenting full, spreading foliage early in tree life.
Classification: Cercis canadensis ‘Gold Crown’.
Parentage: Unknown; believed to be a seedling variation, mutation or adaptation of Cercis canadensis.
Shape: Deciduous tree, nearly isodiametric, with rounded crown and spreading branches, its breaks (stems) being three to four inches or closer than presumed parentage of Cercis canadensis. See FIGS. 1, 3.
Height: Six (6 ft.) feet, increasing an average of twelve (12) inches per year, slowing with age but expected to reach approximately fifteen (15) feet at maturity, as typical of Cercis canadensis. See FIGS. 1, 3.
Spread: Five and one-half (5½ ft.) feet increasing an average of one to one and one-half (1-1.5 ft/yr) feet per year, slowing with age but expected to reach twelve to fifteen, and occasionally eighteen (12-15, 18 ft.)] feet at maturity, as typical of Cercis canadensis. See FIG. 1.
Trunk: Smooth bark, even in young trees, reddish-brown to brown-black in color similar to and as typical of Cercis canadensis, with tan or light-brown, lenticils abundant on both trunk and main branches. Caliper size is one (1 in.) inch in four (4) years, measured at ground level. See FIGS. 2, 8.
Branches: New growth is white (RHS 145A) to yellow (RHS 145C), browning to typical Cercis canadensis branch coloration as it matures. Develops more branches than typical for Cercis canadensis, however, in a zig-zag growth patern, the depicted example having developed twenty-eight (28) branches off its main trunk within three to four (3 to 4 yrs.) years. The effect is full, spreading foliage in young trees. See FIGS. 3-5, 8-10.
Leaves:
   Shape.—Simple, heart-shaped, two (2 in.) inches long and two (2 in.) inches wide in mature leaves. Glabrous, ovate-deltoid, entire margins, slightly acuminate apices, cordate bases and veined axils on the underside. See FIGS. 4-7.
   Color.—The spring leaf coloration has whitish-yellow (RHS 145A) (FIGS. 1, 4, 5, 7) overtones, occasionally mottled (variegate) green and white chimera which is unstable and may be reversed. Summer leaf coloration on new growth exhibits the spring whitish-yellow (RHS 145A) (FIG. 3), 80% of which changing to light yellow over 95% of their surfaces, the remaining 5% of leaf being dull green normal for Cercis canadensis. Remaining 20% of leaves are 80% the dull green normal for Cercis canadensis, while 20% are light yellow (RHS 145C). Fall coloration is yellow (RHS 145C).
Petiole: Moderate Yellowish pink (RHS N170D) in spring, maturing in summer to the reddish brown normal for Cercis canadensis. Sized: average one thirty-second ( 1/32 in.) inch in diameter; average one (1 in.) inch in length, with extremes of two (2 in.) inches in length. Surface texture smooth. Can occur every one-half (½ in.) inch. See FIGS. 6, 7.
Winter buds: Typical of redbuds one-eighth (⅛ in.) inch or less in length, flattened and somewhat appressed; one or more often present. Color normal for Cercis canadensis.
Flowering: Unknown; expected to be perfect, reddish purple in bud, opening to rosy pink with purplish tinge, inflorescent. Size: one-half (½ in.) inch long borne on a one-half (½ in.) inch pedicel, four to eight (4 to 8) together, fascicle or racemose. Blooms in early spring (early March to early April in Oklahoma) typical of Cercis canadensis redbud trees.
Reproductive organs: Unknown, since flowering unknown. Expected to be typical of Cercis canadensis redbud trees.
Pollen: Unknown, since flowering unknown. Expected to be typical of Cercis canadensis redbud trees.
Fruit: None yet observed; expected to be expected to be very similar to other Cercis canadensis varieties, having true pods (legume), brown, two to three (2 in. to 3 in.) long, one-half (½ in.) inch wide; reddish to green prior to maturity.
Disease resistance: Typical for redbud trees.
Climate: Weather tolerant in Zones 5-9 (USDA hardness guidelines). Typical of redbud trees in that it has low chlorophyl and needs shade, at least when young.
Comparison to other cercis canadensis varieties:
   Crown.—Similar rounded crown.
   Trunk.—Similar smooth, even in young trees, color reddish-brown normal for Cercis canadensis, with lenticils present but not prominent.
   Branches.—Different, producing multiple branches every three to four (3″-4″) inches, thereby presenting full, spreading foliage even in young trees.
   Flowers.—Expected to be similar in color, identical in size.
   Reproductive organs.—Similar monoecious imperfect, pollen same color as blooms.
   Fruit.—Expected to be similar or identical.
   Spring color.—Distinctive whitish-yellow (RHS 145A) and occasionally mottled (variegate) green and white chimera which is unstable and may be reversed, turning to predominately light yellow (RHS 145B) during summer and yellow (RHS 145C) during fall.
   Leaf shape and size.—Similar, simple, cordate-accuminate with entire margins, but smaller (2″×2″) than typical redbud leaves, which are three to four (3″-4″) inches wide and long.
   Hardiness.—Similar weather tolerance to Zones 5 -9. Needs shaded environment.

I claim:

1. A new and distinct variety of redbud tree named “Gold Crown”, as described and illustrated herein.
